(19) United States
(12) Patent Application Publication
(10) Pub. No.: US 2025/0228494 A1
(43) Pub. Date: Jul. 17, 2025

(54) A COMPUTING DEVICE, METHOD AND APPARATUS FOR PREDICTING ACNE PROPOERTIES FOR KERATIN MATERIAL OF A HUMAN SUBJECT

(71) Applicant: L'OREAL, Paris (FR)

(72) Inventors: Shuting LI, Shanghai (CN); Zhijun ZHOU, Shanghai (CN); Hequn WANG, Shanghai (CN)

(73) Assignee: L'OREAL, Paris (FR)

(21) Appl. No.: 18/851,433

(22) PCT Filed: Mar. 30, 2022
(86) PCT No.: PCT/CN2022/084011

§ 371 Date: Sep. 26, 2024

(51) Int. Cl. A61B 5/00; G06V 10/44; G06V 10/764

(52) U.S. Cl. CPC A61B 5/445; A61B 5/0033; G06V 10/44; G06V 10/764

(57) ABSTRACT

A computing device, a method, an apparatus and a computer readable medium for predicting acne properties for keratin material of a human object are disclosed. The computing device (100) comprises: acquiring unit (101) including computational circuitry configured to acquire one or more digital images of a region of keratin material of the human subject; extracting unit (102) including computational circuitry configured to extract a plurality of feature data related to acne among all pores from one or more digital images of said region of keratin material of the human subject; classification unit (103) including computational circuitry configured to classify said extracted feature data; and display unit (104) including computational circuitry configured to display said classified results; wherein said classified results are acne proneness information or acne frequency information.

### TECHNICAL FIELD

The disclosure relates to a computing device, a method and an apparatus for predicting acne properties for keratin material of a human object. More specifically, the disclosure relates to predicting acne prone information and acne frequency information.

### BACKGROUND

It is the most important to keep keratin material healthy. However, more and more people suffer from keratin material acne because of environment, pressure and keratin material sensitive and so on. There are two problems to be existed for people all the time. The first problem is that it is difficult to make the classification of “more acne prone keratin material” vs “less acne prone keratin material”; the second problem is that it is difficult to know individual “acne frequency history”, especially to recall an acne history frequency within any time period (e.g. last month, last year, year 2005, last February).

In order to solve such two problems, other Al skin diagnosis system on market has been developed based on facial image at normal scale (not micro), e.g. Yuesai 4T system (Skinrun) and clinical conventional evaluation methodology to differentiate acne prone skin has been developed, which is based on dermatologist assessment by observing in persons with combining the acne history check. However, such current solutions also have some disadvantages, such as the combination of dermatologist diagnostic and self-claim acne history record are needed to make an acne prone and the person is needed to record the acne frequency in vey detail. Such current solutions depend on clinical experts, self-claim acne history record and the acne frequency record, so are not easy to be used for ordinary people.

Therefore, there is a need to predict acne prone information and acne frequency information in an easy and efficient way.

### SUMMARY

The summary is provided to introduce a selection of concepts in a simplified form that are further described below in detailed description. This summary is not intended to identify key features or essential features of the claimed subject matter, nor is it intended to be used to limit the scope of the claimed subject matter.

Various aspects and features of the disclosure are described in further detail below.

According to a first aspect of the disclosure, there is provided a computing device for predicting acne properties for keratin material of a human object, the computing device comprises acquiring unit including computational circuitry configured to acquire one or more digital images of a region of keratin material of the human subject; extracting unit including computational circuitry configured to extract a plurality of feature data related to acne among all pores from one or more digital images of said region of keratin material of the human subject; classification unit including computational circuitry configured to classify said extracted feature data; and display unit including computational circuitry configured to display said classified results; wherein said classified results are acne proneness information or acne frequency information.

In an embodiment of said first aspect, the computing device also comprises data storage including computational circuitry configured to store said digital images.

In an embodiment of said first aspect, said acquiring unit is selected from a microscope, said microscope is preferably a reflectance confocal microscopy.

In an embodiment of said first aspect, said classification unit includes computational circuitry configured to classify a first set of feature data from said extracted feature data by a first machine learning model to obtain a result that said classified results are acne proneness information.

In an embodiment of said first aspect, said classification unit includes computational circuitry configured to classify a second set of feature data from said extracted feature data by a second machine learning model to obtain a result that said classified results are acne frequency information.

In an embodiment of said first aspect, said first machine learning model is trained by obtaining clinical assessment data related to acne for a plurality of sampled human objects; obtaining self-claim data of acne history of said plurality of sampled human objects; acquiring one or more digital images of a region of keratin material of said plurality of sampled human objects; extracting a plurality of feature data related to acne among all pores from one or more digital images of said region of keratin material of said plurality of sampled human subjects; and building said first machine learning model by using said clinical assessment data, said self-claim data of acne history and a first set of feature data from said extracted feature data.

In an embodiment of said first aspect, said second machine learning model is trained by obtaining self-claim data of acne history of said plurality of sampled human objects; acquiring one or more digital images of a region of keratin material of said plurality of sampled human objects; extracting a plurality of feature data related to acne among all pores from one or more digital images of said region of keratin material of said plurality of sampled human subjects; and building said second machine learning model by using said self-claim data of acne history and a second set of feature data from said extracted feature data.

In an embodiment of said first aspect, said first set of feature data comprises ratio of hyper-keratinization follicles.

In an embodiment of said first aspect, said second feature data comprises ratio of hyper-keratinization follicles, ratio of follicles with thick keratinized border and ratio of follicles with inner keratin content.

In an embodiment of said first aspect, ratio of hyper-keratinization follicles is obtained by manually counting the hyper-keratinized follicles among all follicles or by auto-segmentation algorithms.

According to a second aspect of the disclosure, there is provided a method for predicting acne properties for keratin material of a human object, the method comprises: acquiring one or more digital images of a region of keratin material of the human subject; extracting a plurality of feature data related to acne among all pores from one or more digital images of said region of keratin material of the human subject; classifying said extracted feature data; and displaying said classified results; wherein said classified results are acne proneness information or acne frequency information.

In an embodiment of said second aspect, said method also comprises storing said digital images.

In an embodiment of said second aspect, said acquiring one or more digital images of a region of keratin material of the human subject is embodied by a microscope, said microscope is preferably a reflectance confocal microscopy.

In an embodiment of said second aspect, said classifying said extracted feature data also comprises classifying a first set of feature data from said extracted feature data by a first machine learning model to obtain a result that said classified results are acne proneness information.

In an embodiment of said second aspect, said classifying said extracted feature data also comprises classifying a second set of feature data from said extracted feature data by a second machine learning model to obtain a result that said classified results are acne frequency information.

In an embodiment of said second aspect, said first machine learning model is trained by obtaining clinical assessment data related to acne for a plurality of sampled human objects; obtaining self-claim data of acne history of said plurality of sampled human objects; acquiring one or more digital images of a region of keratin material of said plurality of sampled human objects; extracting a plurality of feature data related to acne among all pores from one or more digital images of said region of keratin material of said plurality of sampled human subjects; and building said first machine learning model by using said clinical assessment data, said self-claim data of acne history and a first set of feature data from said extracted feature data.

In an embodiment of said second aspect, said second machine learning model is trained by obtaining self-claim data of acne history of said plurality of sampled human objects; acquiring one or more digital images of a region of keratin material of said plurality of sampled human objects; extracting a plurality of feature data related to acne among all pores from one or more digital images of said region of keratin material of said plurality of sampled human subjects; and building said second machine learning model by using said self-claim data of acne history and a second set of feature data from said extracted feature data.

In an embodiment of said second aspect, said first set of feature data comprises ratio of hyper-keratinization follicles.

In an embodiment of said second aspect, said second feature data comprises ratio of hyper-keratinization follicles, ratio of follicles with thick keratinized border and ratio of follicles with inner keratin content.

In an embodiment of said second aspect, ratio of hyper-keratinization follicles is obtained by manually counting the hyper-keratinized follicles among all follicles or by auto-segmentation algorithms.

According to a third aspect of the disclosure, there is provided a computer readable medium having stored thereon instructions that when executed cause a computing device to perform the above-mentioned method.

According to a fourth aspect of the disclosure, there is provided an apparatus of predicting acne properties for keratin material of a human object, the apparatus comprises means for performing the above-mentioned method.

According to the disclosure, the inventive solution has highly improved the existing solutions, and any users can classify acne prone skin with just imaging method without dermatologist expertise. Also any users can classify a person's recent acne frequency history without knowing or asking them to record and track by themselves.

### BRIEF DESCRIPTION OF THE DRAWINGS

The above and other aspects, features, and benefits of various embodiments of the disclosure will become more fully apparent, by way of example, from the following detailed description with reference to the accompanying drawings, in which like reference numerals or letters are used to designate like or equivalent elements. The drawings are illustrated for facilitating better understanding of the embodiments of the disclosure and not necessarily drawn to scale, in which:

FIG. 1 illustrates a block diagram of a computing device in accordance with a first aspect of the present disclosure;

FIG. 2 (a) illustrates screenshot of overview image of hair follicles with acne prone keratin material;

FIG. 2 (b) illustrates screenshot of local images of hair follicles with acne prone keratin material;

FIG. 3 illustrates a flowchart of a method of predicting acne properties for keratin material of a human object in accordance with a second aspect of the present disclosure;

FIG. 4 illustrates a flowchart of a method of predicting acne proneness information for keratin material of a human object in accordance with a second aspect of the present disclosure;

FIG. 5 illustrates a flowchart of building a first machine learning model in accordance with the present disclosure;

FIG. 6 illustrates an example of building said first machine learning model by using a decision tree;

FIG. 7 illustrates a flowchart of building a second machine learning model in accordance with the present disclosure;

FIG. 8 illustrates an example of building said second machine learning model by using a decision tree.

### DETAILED DESCRIPTION

Embodiments herein will be described in detail hereinafter with reference to the accompanying drawings, in which embodiments are shown. These embodiments herein may, however, be embodied in many different forms and should not be construed as being limited to the embodiments set forth herein. The elements of the drawings are not necessarily to scale relative to each other. Like numbers refer to like elements throughout.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meanings as commonly understood. It will be further understood that a term used herein should be interpreted as having a meaning consistent with its meaning in the context of this specification and the relevant art and will not be interpreted in an idealized or overly formal sense unless expressly so defined herein.

The present technology is described below with reference to block diagrams and/or flowchart illustrations of methods, apparatus (systems) and/or computer program products according to the present embodiments. It is understood that blocks of the block diagrams and/or flowchart illustrations, and combinations of blocks in the block diagrams and/or flowchart illustrations, may be implemented by computer program instructions. These computer program instructions may be provided to a processor, controller or controlling unit of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

Accordingly, the present technology may be embodied in hardware and/or in software (including firmware, resident software, micro-code, etc.). Furthermore, the present technology may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that may contain, store, communicate, propagate, or transport the program for use by or in connection with the instruction execution system, apparatus, or device.

Embodiments herein will be described below with reference to the drawings.

The inventive concept of the disclosure is that the inventive solution is based on imaging device/tool, to observe and segment the hyper-keratinization signal of skin pores of healthy looking skin of a consumer then identify whether they have “more acne prone” or “less acne prone” skin and identify their acne frequency via pre-built machine learning prediction models.

Therefore, the disclosure is direct to identify whether people have “acne prone” skin/or they are acne prone skin population base on imaging acquisition (e.g. confocal microscopy) and also is directed to classify people “acne frequency” base on imaging acquisition (e.g. confocal microscopy).

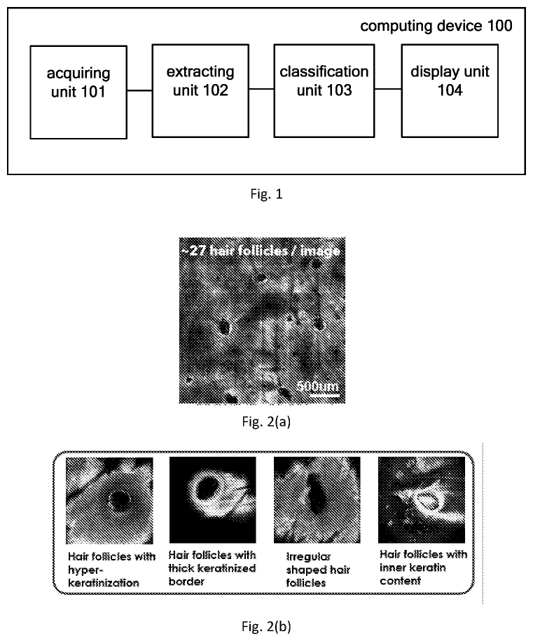

FIG. 1 illustrates a block diagram of a computing device 100 in accordance with a first aspect of the present disclosure. The computing device 100 comprises acquiring unit 101, extracting unit 102, classification unit 103 and display unit 104 which can be communicated with each other via wireless or wire connection. Acquiring unit 101 is to acquire one or more digital images of a region of keratin material of the human subject. Such acquiring unit 101 can be a microscopy such as it is preferably a reflectance confocal microscopy.

A microscopy (Reflectance confocal microscopy (RCM) can be one solution validated by clinical study) which can demonstrate skin pore/follicle 3D imaging on facial area. Principle of the RCM: it is a noninvasive imaging technique that has been utilized on recognition of keratin signal and its distribution within the epidermis, which measures structure of sublayers of skin with cellular level resolution. “Hyper-keratin around hair follicle” provides strong endogenous contrast due to refractive index mismatch versus other content in skin tissue. Normal follicle is a round shape black ‘pore’ which shouldn't have high density keratin signal while the keratinized pores as early sign of acne are will further develop into acne. It is further proved that micro level keratin signs around follicle on a healthy looking skin area from a consumer can links directly with their acne proneness.

Said acquiring unit 101 comprises computational circuitry to obtain images of epidermal level of face area. In a preferred embodiment, the computing device 100 also comprises data storage to store said images. Extracting unit 102 can extract feature data, such as hyper-keratinization signals of multiple follicles/skin pores. Classification unit 103 includes computational circuitry configured to classify said extracted feature data. Display 104 can be any display including computational circuitry configured to display on a graphical user interface one or more instances of the predicted acne data of keratin material. The predicted results can be acne proneness information or acne frequency information.

Computing device 100 can be, for example, a server of a service provider, a device associated with a client (e.g, a client device), a system on a chip, and/or any other suitable computing device or computing system. In various implementations, computing device 100 can take a variety of different configurations. For example, computing device 100 can be implemented as a computer-like device including a personal computer, desktop computer, multi-screen computer, laptop computer, netbook, and the like. Computing device 100 can also be implemented as a mobile device-like device that includes mobile devices such as mobile phones, portable music players, portable gaming devices, tablet computers, multi-screen computers, and the like. Computing device 100 can also be implemented as a television-like device that includes a device having or connected to a generally larger screen in a casual viewing environment. These devices include televisions, set-top boxes, game consoles, and the like.

In an embodiment, computational circuitry includes, among other things, one or more computing devices such as a processor (e.g., a microprocessor, a quantum processor, qubit processor, etc.), a central processing unit (CPU), a digital signal processor (DSP), an application-specific integrated circuit (ASIC), a field programmable gate array (FPGA), and the like, or any combinations thereof, and can include discrete digital or analog circuit elements or electronics, or combinations thereof. In an embodiment, computational circuitry includes one or more ASICs having a plurality of predefined logic components. In an embodiment, computational circuitry includes one or more FPGAs, each having a plurality of programmable logic components.

In an embodiment, computation circuitry includes one or more electric circuits, printed circuits, flexible circuits, electrical conductors, electrodes, cavity resonators, conducting traces, ceramic patterned electrodes, electro-mechanical components, transducers, and the like.

In an embodiment, computational circuitry includes one or more components operably coupled (e.g., communicatively, electromagnetically, magnetically, ultrasonically, optically, inductively, electrically, capacitively coupled, wirelessly coupled, and the like) to each other. In an embodiment, circuitry includes one or more remotely located components. In an embodiment, remotely located components are operably coupled, for example, via wireless communication. In an embodiment, remotely located components are operably coupled, for example, via one or more communication modules, receivers, transmitters, transceivers, and the like.

In an embodiment, computation circuitry includes memory that, for example, stores instructions or information. Non-limiting examples of memory include volatile memory (e.g., Random Access Memory (RAM), Dynamic Random Access Memory (DRAM), and the like), non-volatile memory (e.g., Read-Only Memory (ROM), Electrically Erasable Programmable Read-Only Memory (EEPROM), Compact Disc Read-Only Memory (CD-ROM), and the like), persistent memory, and the like. Further non-limiting examples of memory include Erasable Programmable Read-Only Memory (EPROM), flash memory, and the like. In an embodiment, memory is coupled to, for example, one or more computing devices by one or more instructions, information, or power buses. In an embodiment, computational circuitry includes one or more databases stored in memory. In an embodiment, computational circuitry includes one or more look-up tables stored in memory.

In an embodiment, computational circuitry includes one or more computer-readable media drives, interface sockets, Universal Serial Bus (USB) ports, memory card slots, and the like, and one or more input/output components such as, for example, a graphical user interface, a display, a keyboard, a keypad, a trackball, a joystick, a touch-screen, a mouse, a switch, a dial, and the like, and any other peripheral device. In an embodiment, computational circuitry includes one or more user input/output components that are operably coupled to at least one computing device configured to control (electrical, electromechanical, software-implemented, firmware-implemented, or other control, or combinations thereof) at least one parameter associated with, for example, determining one or more tissue thermal properties responsive to detected shifts in turn-on voltage.

In an embodiment, computational circuitry includes electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs.

The computing device 100 in accordance with a first aspect of the present disclosure can provide acne information or acne frequency quickly, and deliver fast result and a consistent classification result.

In order to class acne proneness information or acne frequency information, two machine learning models are established beforehand. During establishing a first machine learning model and a second machine learning model, a plurality of parameters are loaded. Table 1 illustrates a table of example parameters loaded in the machine learning. As shown in table 1, parameters such as hyper-keratinized percent, thicker percent, irregular percent, inner content percent, stratum corneum follicle top3 mean diameter and so on are obtained from microscopy. However the other parameters are not selected by the model which means they are not key factors. Only the parameters from the microscope are found to be most important for the classification.

TABLE 1

| | | | | | | | | | Stratum |
|---|---|---|---|---|---|---|---|---|---|
| | | | | | | | Inner | | corneum |
| | | Hyper- | Thicker | | Irregular | | content | Inner | follicle |
| volunteer | Acne prone | keratinzed | follicle | Thicker | follicle | Irregular | follicle | content | top3 mean |
| number | class | percent | number | percent | number | percent | number | percent | diameter |
| 6 | 0(less prone) | 35.90% | 6 | 15.38% | 7 | 17.95% | 1 | 2.56% | 77.52 |
| 11 | 0(less prone) | 7.69% | 3 | 5.77% | 4 | 7.69% | 5 | 9.62% | 67.26 |
| 34 | 0(less prone) | 29.27% | 4 | 9.76% | 5 | 12.20% | 1 | 2.44% | 44.46 |
| 43 | 0(less prone) | 35.90% | 5 | 12.82% | 10 | 25.64% | 4 | 10.26% | 101.17 |
| 15 | 1(more prone) | 58.00% | 14 | 28.00% | 13 | 26.00% | 2 | 4.00% | 81.64 |
| 18 | 1(more prone) | 54.35% | 15 | 32.61% | 13 | 28.26% | 6 | 13.04% | 174.56 |
| 19 | 1(more prone) | 48.48% | 18 | 27.27% | 9 | 13.64% | 8 | 12.12% | 109.95 |
| 22 | 1(more prone) | 45.95% | 10 | 27.03% | 9 | 24.32% | 9 | 24.32% | 109.69 |

FIG. 2 (a) illustrates screenshot of overview image of hair follicles with acne prone keratin material. As shown in FIG. 2(a), by such acquiring unit which can be a microscopy preferably a reflectance confocal microscopy, digital images with hair follicles can be obtained. FIG. 2 (b) illustrates screenshot of local images of hair follicles with acne prone keratin material. Hair follicles with hyper-keratinization, hair follicles with thick keratinized border, irregular shaped hair follicles and hair follicles with inner keratin content are shown in FIG. 2(b). From FIG. 2(b), acne prone keratin material also found a higher rate of hyper-keratinized hair follicle, higher rate of thick keratinized border more irregular shaped hair follicle, and more hair follicles with inner keratin contents than less acne prone skin. One or more of such parameters are found to be most important for the classification for acne proneness information and acne frequency information.

For the machine learning models, same parameters (raw data example as table 1) are loaded in the machine learning model training for the two different models, while it is found that the auto selected parameters for the two different models are different. In an embodiment, for the first machine learning to classify acne proneness information, key parameter is ratio of hyper-keratinized follicle. In an embodiment, for the second machine learning to classify acne frequency information, key parameters are ratio of hyper-keratinized follicle, ratio of follicles with thicken keratinized border, ratio of follicles with inner keratin content. Of course, these parameters are only examples and the disclosure is not limited to such parameters. Such parameters auto-selected can be varied.

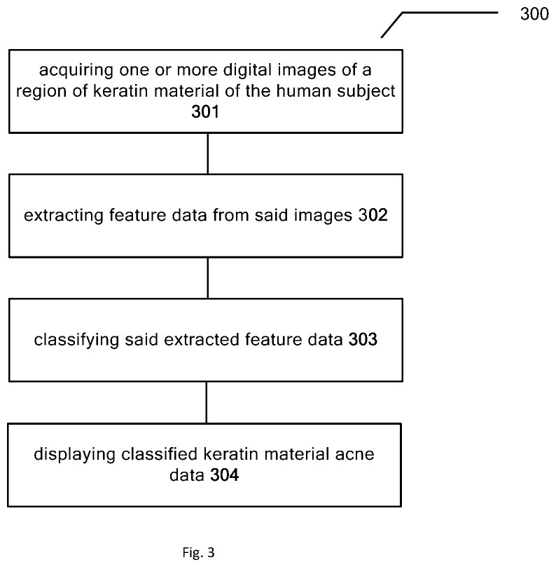

FIG. 3 illustrates a flowchart of a method 300 of predicting acne properties for keratin material of a human object in accordance with a second aspect of the present disclosure. At step 301 of said method 300, one or more digital images of a region of keratin material of the human subject is acquired by imaging acquisition device. Such imaging acquisition device can be a device such as reflectance confocal microscopy which comprises computational circuitry to obtain images of epidermal level of face area. Then, feature data, such as hyper-keratinization signals of multiple follicles/skin pores is extracted from said images as shown at step 302. Then, at step 303, said extracted feature data is classified. Finally, at step 304, one or more instances of the classified acne data of keratin material is displayed on a graphical user interface. The classified results can be acne proneness information or acne frequency information.

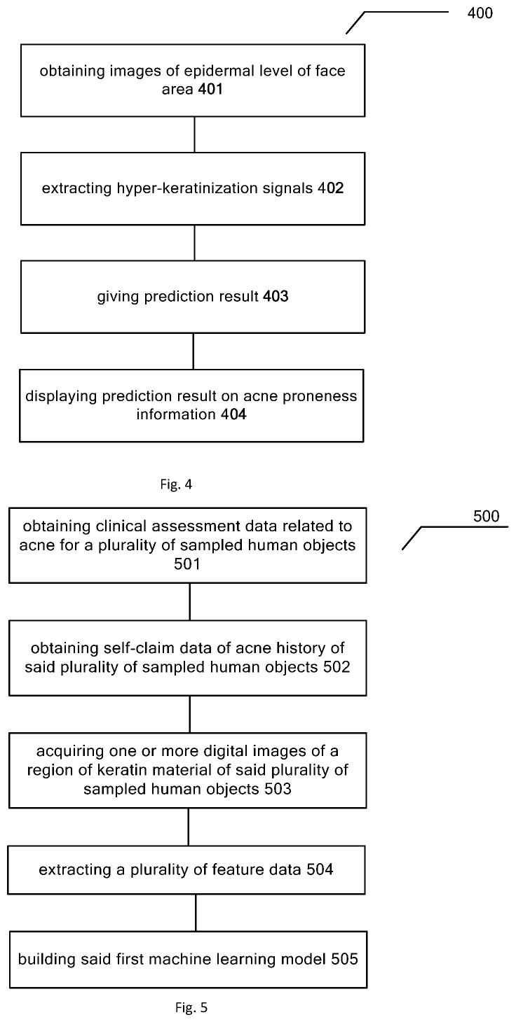

FIG. 4 illustrates a flowchart 400 of a method of predicting acne proneness information for keratin material of a human object in accordance with a second aspect of the present disclosure. As shown in FIG. 4, by imaging acquisition device/tool such as reflectance confocal microscopy, images of epidermal level of face area are obtained as shown at step 401. Then, hyper-keratinization signals of multiple follicles/skin pores are extracted at step 402. The inventors have found the signal of hyper-keratinized pore on the person's facial area is highly correlated to clinical classification of “acne prone skin” and acne history of this person. Based on this, the inventors developed a first machine learning model to classify keratin material type into “more acne prone” or “less acne prone” skin based on the obtained images.

For any new consumer, we will need them to take image acquisition and our system can give prediction result on whether he/she is “more acne prone” or “less acne prone” keratin material based on the parameter “ratio of hyper-keratinization follicles” via the first machine learning models shown at step 403. At last, such prediction result on acne proneness information is displayed as shown at step 404. Of course, input parameter for classifying acne proneness information is only examples and the disclosure is not limited to such parameter.

Comparing to classical clinical assessment on the “acne proneness” classification which needs dermatologist expertise face to face observation with consumer and combining with consumer self-claimed acne history to draw a conclusion, the invention is an Al based automatic system delivering fast and consistent classification result for even non-clinical-experts to use easily. It can work as counter device for brands, or lab device/system.

In addition to classify keratin material type into “more acne prone” or “less acne prone”, the technical solution of method 300 in FIG. 3 can also classify “acne frequency” based on some input parameters via a second machine learning model as detailed in below. In an embodiment, such input parameters for classifying “acne frequency” comprise ratio of hyper-keratinization follicles, ratio of follicles with thick keratinized border and ratio of follicles with inner keratin content. Of course, these parameters are only examples and the disclosure is not limited to such parameters.

FIG. 5 illustrates a flowchart 500 of building a first machine learning model in accordance with the present disclosure. As noted in above, the first machine learning model is trained beforehand to classify “more acne prone” keratin material or “less acne prone” keratin material. During establishing said first machine learning model, clinical assessment data related to acne for a plurality of sampled human objects is obtained at step 501. Such clinical assessment can be dermatologist diagnostic of “more acne prone keratin material” or “less acne prone keratin material”. Then, at step 502, self-claim data of acne history of said plurality of sampled human objects is obtained. For example, self-claim of acne history can be an average status among past 1 year related to acne. By such clinical assessment data and self-claim of acne history, a criteria regarding “acne prone skin=1” and “less acne prone skin=0” can be specified. Then, at step 503, one or more digital images of a region of skin of said plurality of sampled human objects are acquired. In an example, digital images can be images of epidermal level of face area.

Then, at step 504, a plurality of feature data related to acne among all pores from one or more digital images of said region of keratin material of said plurality of sampled human subjects are extracted.

Finally, at step 505, said first machine learning model is built by using said clinical assessment data, said self-claim data of acne history and a first set of feature data from said extracted feature data. In an example, parameter “ratio of hyper-keratinization follicles” as a fist set of feature data is extracted, since it is proved that the signal of hyper-keratinized pore on the person's facial area is highly correlated to clinical classification of “acne prone skin”. In an example, the first machine learning model for classification ‘acne proneness’ is built with ‘scikit-learn’ package from OpenCV[1] using the data from steps 501, 502, 504.

For one clinical trail (N=15 subjects), the test showed that the size of the follicles in acne-prone skin range is larger than less acne prone skin follicle's size. Acne prone keratin material also found a higher rate of hyper-keratinized hair follicle, higher rate of thick keratinized border more irregular shaped hair follicle, and more hair follicles with inner keratin contents than less acne prone skin as shown FIG. 2(b). RCM measurement results also showed a high correlation with clinical signs of inflammatory acnes, PIE (Post-inflammation erythema), PIH (Post-inflammation hyperpigmentation) and self-claimed historical acne frequency. It is also found the ratio of hyper-keratinized hair follicles and the thick keratin border decreases after 4-week treatment of a face serum product.

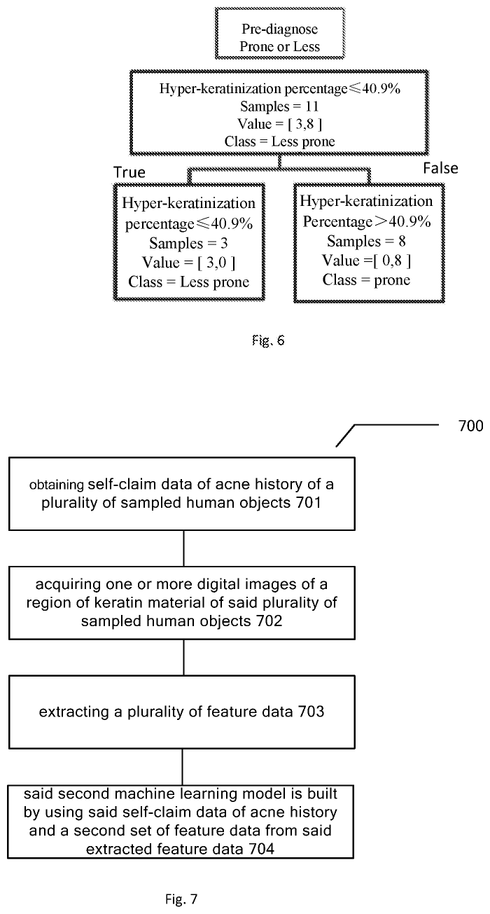

FIG. 6 illustrates an example of building said first machine learning model by using a decision tree. As shown in FIG. 6, it is Model 1—Acne Prone Skin vs Less Acne Prone Skin machine learning model. In an example, by using “hyper-keratinization percentage” as INPUT, OUTPUT is “acne prone skin” or “less acne prone keratin material”. In an example, a predetermined value for hyper-keratinization percentage is set to 40.9% and the predetermined value can be varied depended on actual need. If hyper-keratinization percentage >4.9%, Classification=“acne prone keratin material” as right branch. If hyper-keratinization percentage <=4.9%, Classification=“less acne prone skin” as left branch.

For a new user, by using imaging acquisition device/tool (e.g. Reflectance confocal microscopy) to obtain skin images of epidermal level of face area with hyper-keratinization signals of multiple follicles/skin pores. Then from the image, the user can obtains the VIP parameters (e.g. ‘keratinized follicle ratio’) for acne prone classification by processing the images either by manually count the hyper-keratinized follicle among all follicles (e.g. 45% follicles of total 30 follicles are keratinized), or by auto-segmentation algorithms to get the same parameters. The result from the VIP parameters is input to a “decision tree” example as shown in FIG. 6, output is the classification of “acne proneness”.

By such first machine learning model, an acne prone skin can be predicted quickly and a fast and consistent classification result can be delivered.

FIG. 7 illustrates a flowchart of building a second machine learning model in accordance with the present disclosure. As noted above, the second machine learning model is trained beforehand to classify “acne frequency”. During the second machine learning model, self-claim data of acne history of a plurality of sampled human objects is obtained at step 701. For example, self-claim of acne history can be an average status among past 1 year related to acne. Then, at step 702, one or more digital images of a region of keratin material of said plurality of sampled human objects are acquired. In an example, digital images can be images of epidermal level of face area.

Then, at step 703, a plurality of feature data related to acne among all pores from one or more digital images of said region of keratin material of said plurality of sampled human subjects are extracted.

Finally, at step 704, said second machine learning model is built by using said self-claim data of acne history and a second set of feature data from said extracted feature data. In an example, parameters “ratio of hyper-keratinization follicles”, “ratio of follicles with thick keratinized border” and “ratio of follicles with inner keratin content” as a second set of feature data are extracted, since it is proved that “ratio of hyper-keratinization follicles”, “ratio of follicles with thick keratinized border” and “ratio of follicles with inner keratin content” are highly correlated to clinical classification of “acne frequency level”. However, said second set of feature data is not limited to parameters “ratio of hyper-keratinization follicles”, “ratio of follicles with thick keratinized border” and “ratio of follicles with inner keratin content” and can be varied depended on actual need. In an example, the second machine learning model for classification ‘acne frequency’ is built with ‘scikit-learn’ package from OpenCV[1] using the data from steps 701, 703.

For one clinical trail (N=15 subjects), individual self-claimed acne frequency level scale can be 0-3 (N=15). It is proved that individual self-claimed acne frequency level has high correlation with instrumental result in “hyperkeratinization” (0.67) and “thick keratinized border percentage” (0.717).

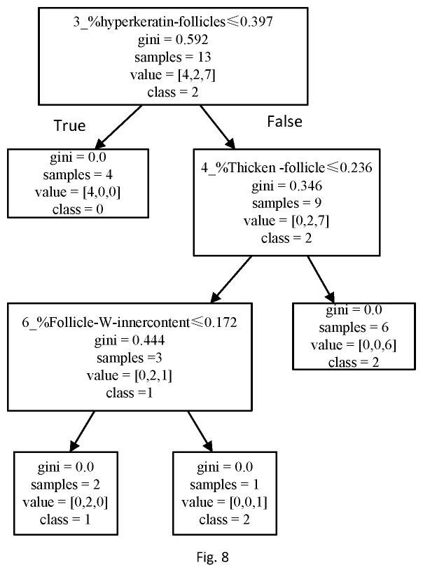

FIG. 8 illustrates an example of building said second machine learning model by using a decision tree. As shown in FIG. 8, it is Model 2—Acne Frequency classification machine learning model, by using “ratio of hyper-keratinization follicles”, “ratio of follicles with thick keratinized border” and “ratio of follicles with inner keratin content” as INPUT, OUTPUT is CLASS=0, CLASS=1, CLASS=2, wherein Class 0=“acne happen less than once every 3 month”, Class 1=“acne happens once very month”; Class2=“acne happens every 2 weeks”. Predetermined values corresponding to “ratio of hyper-keratinization follicles”, “ratio of follicles with thick keratinized border” and “ratio of follicles with inner keratin content” respectively are set to 39.7%, 23.6% and 17.2%. It is noted that corresponding predetermined values corresponding to “ratio of hyper-keratinization follicles”, “ratio of follicles with thick keratinized border” and “ratio of follicles with inner keratin content” are not limited to the above values and can be varied depended on actual need.

In an example, if hyper-keratinization is <=39.7%, then Frequency level=0. If hyper-keratinization is >39.7% and thick keratinized border is <=23.6%, then Frequency level=2. If hyper-keratinization is >39.7%, thick keratinized border is >23.6% and inner keratin content is >17.2%, then Frequency level=2. If hyper-keratinization is >39.7%, thick keratinized border is <23.6% and inner keratin content is <=17.2%, then Frequency level=1.

For a new user, by using imaging acquisition device/tool (e.g. Reflectance confocal microscopy) to obtain skin images of epidermal level of face area with hyper-keratinization signals of multiple follicles/skin pores. Then from the image, the user can obtains the parameters “ratio of hyper-keratinization follicles”, “ratio of follicles with thick keratinized border” and “ratio of follicles with inner keratin content” for acne frequency classification, wherein ratio of hyper-keratinization follicles can be obtained by processing the images either by manually count the hyper-keratinized follicle among all follicles (e.g. 45% follicles of total 30 follicles are keratinized), or by auto-segmentation algorithms. The obtained parameters are input to a “decision tree” example as shown in FIG. 8, output is the classification of “acne frequency”.

By such second machine learning model, an acne frequency information can be predicted quickly and a fast and consistent classification result can be delivered.

Method for implementing the first machine learning model and the second machine learning model of the disclosure is not limited to decision tree method. Instead, they can also employ deep learning method to be established.

By using technical solution, the result will be showed to the consumers to let them know what their “acne prone class” and “acne frequency class”. The invention can be applied to anywhere such as on a counter, where no on-site dermatologist needed and can work any time, immediately after image acquisition or a time period afterwards. Our invention device can be a counter device for brands for consumer to get result before/after application of a cosmetic product. It can also be a personalize tool to guide consumer to select product suitable for their skin condition.

An embodiment of the disclosure may be an article of manufacture in which a non-transitory machine-readable medium (such as microelectronic memory) has stored thereon instructions (e.g., computer code) which program one or more data processing components (generically referred to here as a “processor”) to perform the operations described above. In other embodiments, some of these operations might be performed by specific hardware components that contain hardwired logic (e.g., dedicated digital filter blocks and state machines). Those operations might alternatively be performed by any combination of programmed data processing components and fixed hardwired circuit components.

While the embodiments have been illustrated and described herein, it will be understood by those skilled in the art that various changes and modifications may be made, and equivalents may be substituted for elements thereof without departing from the true scope of the present technology. In addition, many modifications may be made to adapt to a particular situation and the teaching herein without departing from its central scope. Therefore it is intended that the present embodiments not be limited to the particular embodiment disclosed as the best mode contemplated for carrying out the present technology, but that the present embodiments include all embodiments falling within the scope of the appended claims.

1: A computing device for predicting acne properties for keratin material of a human object, the computing device comprising:
an acquiring unit including computational circuitry configured to acquire one or more digital images of a region of keratin material of the human subject;
an extracting unit including computational circuitry configured to extract a plurality of feature data related to acne among all pores from said one or more digital images of said region of keratin material of the human subject;
a classification unit including computational circuitry configured to classify said extracted feature data; and
a display unit including computational circuitry configured to display said classified results;
wherein said classified results are acne proneness information or acne frequency information.

2: The computing device of claim 1, wherein the computing device further comprises data storage to store said digital images.

3: The computing device of claim 1, wherein said acquiring unit is embodied by a microscope.

4: The computing device of claim 1, wherein said classification unit includes computational circuitry configured to classify a first set of feature data from said extracted feature data by a first machine learning model to obtain a result that said classified results are acne proneness information.

5: The computing device of claim 4, wherein said classification unit includes computational circuitry configured to classify a second set of feature data from said extracted feature data by a second machine learning model to obtain a result that said classified results are acne frequency information.

6: The computing device of claim 4, wherein said first machine learning model is trained by the following actions:
obtaining clinical assessment data related to acne for a plurality of sampled human objects;
obtaining self-claim data of acne history of said plurality of sampled human objects;
acquiring one or more digital images of a region of keratin material of said plurality of sampled human objects;
extracting a plurality of feature data related to acne among all pores from one or more digital images of said region of keratin material of said plurality of sampled human subjects; and
building said first machine learning model based on said clinical assessment data, said self-claim data of acne history and a first set of feature data from said extracted feature data.

7: The computing device of claim 5, wherein said second machine learning model is trained by the following actions:
obtaining self-claim data of acne history of said plurality of sampled human objects;
acquiring one or more digital images of a region of keratin material of said plurality of sampled human objects;
extracting a plurality of feature data related to acne among all pores from one or more digital images of said region of keratin material of said plurality of sampled human subjects; and
building said second machine learning model based on said self-claim data of acne history and a second set of feature data from said extracted feature data.

8: The computing device of claim 4, wherein said first set of feature data comprises a ratio of hyper-keratinization follicles.

9: The computing device of claim 5, wherein said second feature data comprises a ratio of hyper-keratinization follicles, a ratio of follicles with thick keratinized border and a ratio of follicles with inner keratin content.

10: The computing device of claim 8, wherein the ratio of hyper-keratinization follicles is obtained by manually counting the hyper-keratinized follicles among all follicles or by auto-segmentation algorithms.

11: A method for predicting acne properties for keratin material of a human object, comprising:
acquiring one or more digital images of a region of keratin material of the human subject;
extracting a plurality of feature data related to acne among all pores from one or more digital images of said region of keratin material of the human subject;
classifying said extracted feature data; and
displaying said classified results;
wherein said classified results are acne proneness information or acne frequency information.

12: The method of claim 11, further comprising storing said digital images.

13: The method of claim 11, wherein said acquiring one or more digital images of a region of keratin material of the human subject is embodied by a microscope.

14: The method of claim 11, wherein classifying said extracted feature data further comprises classifying a first set of feature data from said extracted feature data by a first machine learning model to obtain a result that said classified results are acne proneness information.

15: The method of claim 14, wherein classifying said extracted feature data further comprises classifying a second set of feature data from said extracted feature data by a second machine learning model to obtain a result that said classified results are acne frequency information.

16: The method of claim 14, wherein said first machine learning model is trained by the following actions:
obtaining clinical assessment data related to acne for a plurality of sampled human objects;
obtaining self-claim data of acne history of said plurality of sampled human objects;
acquiring one or more digital images of a region of keratin material of said plurality of sampled human objects;
extracting a plurality of feature data related to acne among all pores from one or more digital images of said region of keratin material of said plurality of sampled human subjects; and
building said first machine learning model based on said clinical assessment data, said self-claim data of acne history and a first set of feature data from said extracted feature data.

17: The method of claim 15, wherein said second machine learning model is trained by the following actions steps:
obtaining self-claim data of acne history of said plurality of sampled human objects;
acquiring one or more digital images of a region of keratin material of said plurality of sampled human objects;
extracting a plurality of feature data related to acne among all pores from one or more digital images of said region of keratin material of said plurality of sampled human subjects; and
building said second machine learning model by-using based on said self-claim data of acne history and a second set of feature data from said extracted feature data.

18: The method of claim 14, wherein said first set of feature data comprises a ratio of hyper-keratinization follicles.

19: A computer readable medium having stored thereon instructions that when executed cause a computing device to perform the method according to claim 11.

20: An apparatus for predicting acne properties for keratin material of a human object, the apparatus comprises means for performing the method according to claim 11.
